(19) United States
(12) Patent Application Publication
(10) Pub. No.: US 2011/0208195 A1
(43) Pub. Date: Aug. 25, 2011

(54) POSITIONING CYLINDER FOR DRILLING WITH A SURGICAL DRILL AND DRILLING JIG AND SYSTEM FOR DRILLING

(75) Inventors: Ady Palti, Baden-Baden (DE); Herbert Hatzlhoffer, Waldbronn (DE)

(73) Assignee: GPI TECHNOLOGY GMBH, Baden-Baden (DE)

(21) Appl. No.: 12/991,532

(22) PCT Filed: May 9, 2008
(86) PCT No.: PCT/EP08/03738

§ 371 Date: May 4, 2011

(51) Int. Cl. A61B 17/16; A61B 17/56

(52) U.S. Cl. 606/80; 606/96

(57) ABSTRACT

The invention relates to a positioning cylinder for a surgical drill, which positioning cylinder is screwed into a drilling jig by means of an external thread, especially for drilling in a jawbone for a dental implant. The positioning cylinder comprises a cylinder base having an axial internal bore and an external thread for screwing it into the drilling jig. The positioning cylinder is designed as a one-piece or integral part and the internal bore is used to guide the drill. The invention further relates to a system consisting of said positioning cylinders and surgical drills.

The invention relates to a positioning cylinder that can be screwed into a drilling jig in order to position and guide a surgical drill when, drilling a hole in a bone, in particular in a jaw hone for the purpose of inserting a tooth implant into the jaw bone or for introducing a pilot hole for a tooth implant into the jaw bone. The invention is further directed to a system for drilling a hole in a bone using a surgical drill with a predefined, drilling depth of the hole in the bone, in particular in a jaw bone for the purpose of inserting a tooth implant into the jaw bone or for introducing a pilot hole for a tooth implant into the jaw bone, with positioning cylinders and surgical drills.

The positioning cylinder according to the invention can be used in all fields of bone surgery. It is described below, without limiting the scope of the invention, using the example of drilled implant holes for oral surgery.

Tooth implants are foreign bodies fitted in the jaw bone. The subsidiary field of dentistry concerned with the implantation of tooth implants into the jaw bone is referred to as implantology. By virtue of their being able to be used as supports for a tooth replacement, tooth implants assume the function of artificial tooth roots. A drilling jig is used to drill the holes into the jaw for the tooth implants.

The technique of replacing a lost tooth with a tooth implant, and with a tooth prosthesis or bridge secured on the latter, has by now become widely accepted. An implant made of a ceramic compound or of metal is anchored in the bone and acts as the implant root, and the artificial crown is secured on the implant. For this purpose, a hole for the implant root has to be introduced into the jaw at the site of the lost tooth. Since the artificial crown is to merge harmoniously into the dentition and since the implant root is to have the greatest possible diameter, in order to better absorb mastication pressure, and since the available bone in the jaw is limited, the position and angular orientation of the hole must be accurately calculated in advance and maintained.

To guarantee this, a drilling jig is usually first set up which, at the predetermined location, has a drill cylinder which is adjusted in terms of angular position and whose internal diameter corresponds to the diameter of a pilot drill for drilling a hole in the jaw. The drilling jig is worn by the patient while the pilot hole is being drilled. This drilling jig can be produced on the bsisis of a model of the patient's jaw or on the basis of data obtained purely by X-ray or computed tomography. Moreover, the information needed to determine the drilling direction and concerning the extent of the jaw bone is obtained by means of computed tomography, with the possibility of different sectional views through the jaw. Other methods used to measure the jaw in order to produce a drilling jig are, for example, bone mapping, bone measurement with a probe, or other measuring methods.

Drilling jigs are therefore aids that make it easier for the implantologist to make a hole in the jaw bone of a patient, into which hole the implant is to be inserted. The drilling jig has a drill hole which is produced on the jaw model and which serves as a guide for the drill when introducing trie hole or pilot hole into the jaw bone. The drilled, hole should have the correct position and single.

Before an implant is inserted into a bone, the bone substance is first prepared using special surgical tools. A preliminary hole, the so-called pilot hole, is often first formed using a relatively thin drill, the preparation depth being guaranteed by depth-limiting elements. Thereafter, the channel of the preliminary hole is drilled wider with the aid of a so-called forming drill and thus acquires the shape necessary for the implant.

Positioning aids are introduced into the drilling jig at the implantation sites and serve to position and guide the surgical drill during drilling. The positioning aids are generally cylinders. One-piece positioning cylinders are in practice secured in the drilling jig by being oast in or polymerized in. The positioning cylinders are used, to drill a pilot hole with a defined depth into the jaw. The drilling direction and the drilling angle are predetermined by the position of the positioning cylinder in the drilling jig. The drilling jig, with the positioning cylinders fitted therein, is worn in the jaw of the patient during the drilling of the pilot hole, and the positioning cylinders are used to guide and limit, the depth of the pilot drill.

Before holes can be drilled in the jaw bone, the gum has to be removed at the corresponding sites. For this purpose, the gum is generally cut open and folded aside. Thereafter, the drilling jig is fitted into the mouth area fitting it. By removal of the gum, however, the mouth area has changed greatly. The drilling jig no longer lies without gaps in the corresponding working area, because the removal of the gum has resulted in hollow spaces. In so-called free-end situations or in cases of complete absence of teeth, this can lead to considerable problems, since the drilling jig is not oriented in its desired position, in particular during the drilling procedure itself. If the intended orientation of the drilling jig is no longer ensured, this can cause incorrect drilling, with the actual position of the hole differing from the desired position of the hole in terms of setting, angle or depth. This can have the effect that the implants do not adopt the desired positions in the jaw bone.

Moreover, the hollow spaces between the jaw bone substance and the drilling jig can have the effect that, during the drilling procedure, the drilling jig is deformed by forces acting on it. The drilling jig can, for example, sag in the proximal direction, or twist on itself.

A drilling jig with two-part positioning cylinders allowing an exact orientation and positioning of the drill is known in the prior art from EP 1 321 107 A1, The latter describes a positioning cylinder that can be screwed, into a drilling jig in order to position and guide a surgical drill when drilling a hole in a bone, in particular in a jaw bone for the purpose of inserting a tooth implant into the jaw bone or for introducing a pilot hole for a tooth implant into the jaw bone, comprising
a cylinder body that extends in an axial direction,
and that has a continuous axial inner bore and
an outer thread for screwing the positioning cylinder into a drilling jig.

The positioning cylinder comprises two combined parts, namely an outer positioning element, which is screwed into the drilling jig, and an inner spacer element, which is screwed into the positioning element. Since the outer part of the two-part positioning cylinders is screwed into the drilling jig, the positioning cylinders can be fitted much more quickly and exactly, because they do not nave to be successively fixed during the hardening of the polymer.

Although good results were able to be achieved with these known positioning cylinders, it was found in practice that they do not optimally satisfy the requirements ana need to be improved.

In the case of the known two-part positioning cylinder that can be screwed into place, a disadvantage in practical application is that two elements are needed. Moreover, the inner spacer element has either an outer right-handed thread or an outer left-handed thread with which it is screwed into the outer positioning element. If a pilot drill with rightward rotation is used, an outer right-handed thread has the effect that, in the possible event of jamming or the occurrence of other forces that are exerted on the spacer element during drilling, the spacer element is moved further in the direction of the bone, which could lead to the drilling jig lifting or to the bone being damaged. By contrast, an outer left-handed thread has the effect that the spacer element, in this case moves away from the jaw bone, which has the disadvantage that the drilling depth decreases, namely by the extent by which the spacer element moves distally out of the drilling jig or the positioning element.

Proceeding from this, the object of the present invention is to make available a positioning cylinder that can be screwed, into a drilling jig and that is easier to use in practice and more reliably ensures that the predefined drilling depth is observed.

According to the invention, the object is achieved by a positioning cylinder with the features of claim 1. Preferred embodiments are set forth in the dependent claims, and in the description given below with reference to associated drawings.

A positioning cylinder according to the invention that can be screwed into a drilling jig in order to position and guide a surgical drill when drilling a hole in a bone, in particular in a jaw bone for the purpose of inserting a tooth implant into the jaw bone or for introducing a pilot hole for a tooth implant into the jetw bone, thus comprises
a cylinder body that extends in an axial direction,
and that has a continuous axial inner bore and
an outer thread for screwing the positioning cylinder into a drilling jig,

and is characterized in that
the positioning cylinder is designed as a one-piece or integral part, and
the inner bore is used to guide the drill.

It has been found that such a positioning cylinder permits optimal drilling in the drilling jig. The reason for this is, on the one hand, that only one part instead of two parts has to be used per drilled hole, that is to say, in contrast to the prior art, no additional spacer element is necessary, and, on the other hand, that the positioning cylinder is fixed, more reliably in the drilling jig. The latter feature is due to the fact that the positioning cylinder according to the invention can have a greater external diameter (ca. 4 mm) than the spacer element of the known positioning cylinder and therefore sits more securely in the jig. The maximum external diameter is in fact limited by the dimensions of the drilling jig, which in turn are predefined by the circumstances of the jaw.

Further advantages are afforded by the possibility of screwing the cylinder into contact with bone, such that the drilling jig can be supported not only in gaps between teeth but also in edentulous situations (so-called free situation). The invention is therefore of great advantage particularly for treating free-end situations or edentulous jaws, since the drilling jig can be supported on the jaw bone in a defined manner and, as a result, the drilling jig is oriented according to the jaw model. However, in order to establish the exact drilling depth, it is not by any means absolutely necessary to screw the positioning cylinder into the drilling jig until contact with bone is made. For example, it can also be positioned over the bone, on the mucosa, on the gum or at a distance therefrom, which is advantageous particularly in the case of newly extracted teeth or of strongly atrophied jaw bone.

The invention is explained in more detail below with reference to illustrative embodiments shown in the figures. The features described can be used singly or in combination with one another to obtain preferred embodiments of the invention.

FIG. 1 shows a side view of a positioning cylinder according to the invention,

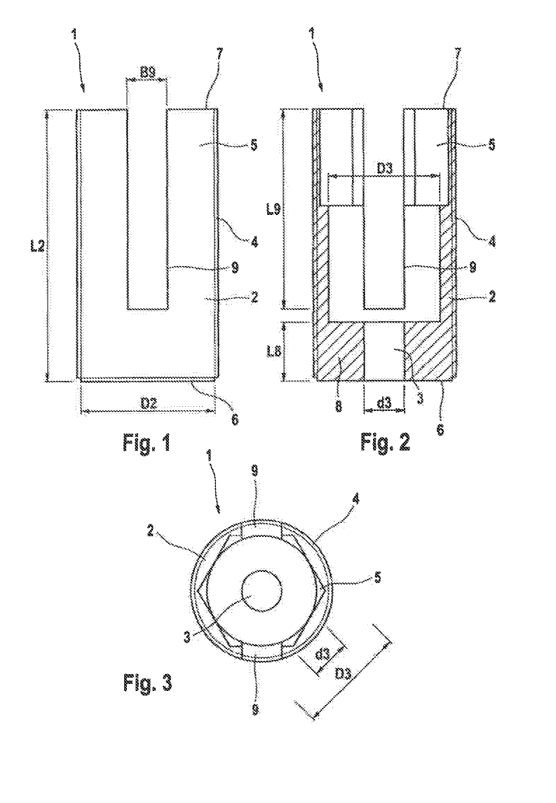

FIG. 2 shows an axial longitudinal section through FIG. 1,

FIG. 3 shows a plan view of the distal end of the positioning cylinder from FIG. 1,

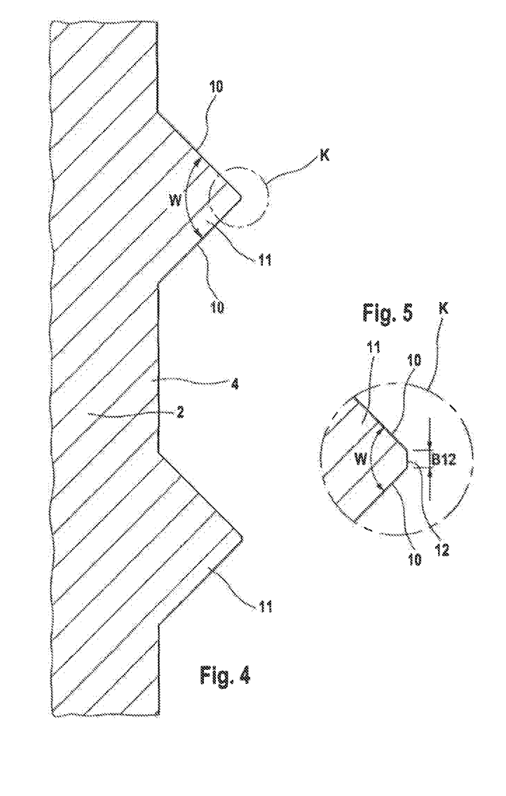

FIG. 4 shows an outer thread of the positioning cylinder from FIG. 1, and

FIG. 5 shows a detail from FIG. 4.

In FIGS. 1 to 5, an illustrative embodiment is shown of a positioning cylinder 1 according to the invention for drilling a hole in a jaw in order to insert a tooth implant. FIGS. 1 to 3 show different views and sections, and FIGS. 4 and 5 show details of the outer thread, of the positioning cylinder 1.

The positioning cylinder 1 comprises a cylinder body 2 that extends in an axial direction and that has a continuous axial inner bore 3. The drill with which a hole is drilled in a jaw bone, using the positioning cylinder 1 and a drilling jig (not shown; into which the positioning cylinder 1 is inserted, is guided through this inner bore 3.

In order to insert and fix the positioning cylinder 1 in a corresponding hole in the drilling jig, the positioning cylinder 1 has an outer thread 4 with which the positioning cylinder 1 is screwed into the drilling jig. Instead of easting or polymerizing the positioning cylinder 1 into the drilling jig, it has proven particularly advantageous to screw it into the drilling jig, as a result of which it is not necessary to wait a long time for the drilling jig to harden after introduction of the positioning cylinder 1, and instead the fitting work can be quickly continued, without waiting, which is advantageous particularly in the case of several positioning cylinders 1 that are introduced together into a drilling jig. For this purpose, the positioning aid 1 is provided with the outer thread 4. It is expedient in this case if the positioning aid 1 additionally has a tool socket 5 into which a tool, for example an Allen key, for example a hexagon, key, can engage in order to screw the positioning aid 1 into the drilling jig. The tool socket 5 is preferably located at the distal end of the cylinder body 2.

The positioning cylinder 1 is designed as a one-piece or integral part, with the inner bore 3 in the cylinder body 2 serving to guide the drill. The cylinder body 2 therefore does not comprise any spacer element inserted, therein for adjusting the distance of the positioning aid 1 from the bone and for guiding the drill, but instead only the cylinder body 2 screwed into the drilling jig, which cylinder body 2 tabes over these functions.

To be able to easily fit the positioning cylinder 1 or the cylinder body 2 to any desired depth or in any desired position in the drilling jig, it is advantageous if the cylinder body 2 has a constant external diameter D2 along its entire length L2 and/or if the outer thread 4 extends over the entire axial outer surface of the cylinder body 2, In some embodiments, it can also be expedient if the cylinder body 2 has a constant external diameter D2 along its entire length L2, except for a small conicaily tapering tip at its proximal end 6. In. FIGS. 1 and 2, the proximal end 6 is at the bottom and the distal end 7 at the top.

The external diameter D2 of the cylinder body 2 is advantageously between 3 mm and 8 mm, preferably between 3.5 mm and 6 mm, particularly preferably between 4 mm and 5 mm. The axial length L2 of the cylinder body 2 is advantageously between 3 mm and 35 mm, preferably between 8 mm said 30 mm, particularly preferably between 10 mm and 25 mm.

At its proximal end 6, the cylinder body 2 has a cylinder base 8 through which the inner bore 3 for guiding the drill is routed. The axial thickness L8 of the cylinder base 8 is advantageously between 0.5 mm and 10.0 mm, preferably between 1.0 mm and 8 mm, particularly preferably between 1.5 mm and 5 mm.

According to another advantageous feature, it is proposed that the cylinder base 8 has an axial thickness L6 that is between 1% and 25%, preferably between 3% and 20%, particularly preferably between 5% and 15% of the axial length L2 of the cylinder body. In advantageous embodiments of the positioning cylinder 1, the diameter of the inner bore 3 in the cylinder base 8 is between 0.3 mm and 5.0 mm, preferably between 0.9 mm and 3 mm, particularly preferably between 1.0 mm and 2.0 mm.

It can be seen in FIGS. 2 and 3 that the inner bore 3 has a greater diameter D3 at the distal end 7 of the cylinder body 2 than the dianieter d3 of the inner bore 3 in the cylinder base 8. This has the advantage that the drill is not guided tightly in the inner, bore 3 along the entire length L2 of the cylinder body 2, but only at the proximal end 6. It has been found that this causes less jamming of the drill if the latter is fitted obliquely into the inner bore 3 or into the positioning cylinder 1, because the drill is not guided along the entire length 2 of the positioning cylinder 1 but only via the axial thickness L8 of the comparatively thinner cylinder base 8. In the space lying above the cylinder base 8, the drill is not guided tightly and can therefore perform lateral deflection movements, however, these lateral deflection movements cannot be of any random amplitude and instead are limited, on the one hand, by the dimensions of the inner bore 3 in the cylinder base 8 and, on the other hand, by the shaft, of the cylinder body 2 lying distaily above the cylinder base 8. In this way, the drill or the drill shank also experiences deflection limitation, at the distal end 7 of the cylinder body 2, and advantageous values of the deflection from the axial center are a maximum of 1° to 5° or 10°.

In an advantageous embodiment, provision is made that the inner bore 3, with the greater diameter D3 at the distal end 7 of the cylinder body 2, extends in the axial direction as far as the distal end of the cylinder base 8. Provision is advantageously made that the inner bore 3 at the distal end 7 of the cylinder body 2 has a diameter D3 which is more than 10%, 20%, 30%, 40%, 50%, 60%, 80%, 100%, 125%, 150% or 200% greater than the diameter d3 of the inner bore 3 in the cylinder base 8 .

At its distal end 7, the cylinder body 2 advantageously has at least one axial longitudinal slot 9 in the wall of the cylinder body 2, which slot 9 extends in the axial proximal direction from the distal end 7 of the cylinder body 2. Such a longitudinal slot 9 can serve as an “access aid”, in order to be able to insert the drill more easily into the positioning cylinder 1 when space is confined in the mouth of a patient. Such slots are therefore particularly advantageous in positioning cylinders 1 that are used in the area of the molars. The longitudinal slots at the distal end of the cylinder body 2 therefore allow the drill to be inserted more easily into the positioning cylinder 1 when space is confined in the mouth.

In order to be able to insert the drill into the axial longitudinal slot 9 from the side, it is advantageous if the width B9 of the longitudinal slot 9 is greater than the diameter d3 of the inner bore 3 of the cylinder body 2 or of the inner bore 3 in the cylinder base 8 (and thus greater than the diameter of the drill, provided). In advantageous embodiments, the width B9 of the axial longitudinal slot 9 is more than 110%, 125%, 150%, 175%, 200%, 250% or 300% of the diameter d3 of the inner bore 3 in the cylinder base 8. Provision is advantageously also made that the width B9 of the axial longitudinal slot 9 is less than 500%, 450%, 300%, 250% or 200% of the diameter d3 of the inner bore 3 in the cylinder base 8. Moreover, in order to permit easier insertion of the drill into the positioning cylinder 1, it is advantageous if the axial longitudinal slot 9 extends along a length L9 that is between 10% and 75%, preferably between 20% and 60%, particularly preferably between 25% and 50% of the axial length L2 of the cylinder body 2.

In advantageous embodiments, provision is made that the positioning cylinder 1 has a plurality of axial longitudinal slots 9. In a preferred embodiment, which permits easy insertion of the drill and also ensures sufficient mechanical stability of the cylinder body 2, two axial longitudinal slots 9 are provided. If a plurality of axial longitudinal slots 9 are provided, it is expedient if they are arranged lying opposite each other in pairs and/or if they each, have the same dimensions.

In advantageous embodiments, the positioning cylinder 1 can be made of plastic, industry steel no. 14301, 14303 or 14305, titanium, titanium nitrite, titanium nitride, zirconium, zirconium oxide or ceramic.

FIGS. 4 and 5 illustrate advantageous embodiments of the outer thread 4. In advantageous embodiments, the external diameter of the outer thread 4 is between 0.01 mm and 1.5 mm, preferably between 0.02 mm and 1.0 mm, particularly preferably between 0.03 mm and 0.5 mm greater than the root diameter of the outer thread 4. The lead and/or the pitch of the outer thread 4 is advantageously between 0.5 mm and 4 mm preferably between 0.8 mm and 2 mm.

A particularly advantageous embodiment of an outer thread 4 is a trapezoid thread according to FIG. 4. Here, the thread turns of the trapezoid thread can advantageously have flanks 10 shaped as pointed cones, of which the flank angle w is advantageously between 40° and 80°, preferably between 50° and 70°. It can be seen in FIG. 5 that the thread, teeth 11 have a flat 12. The width B12 of the fiat 12 is advantageously between 0.03 mm and 0.2 mm, preferably between 0.05 mmm and 0.1 mm.

The positioning cylinder 1 can, with the associated drills, form a system for drilling a hole in a bone using a surgical drill with a predefined drilling depth of the hole in the bone, in particular in a jaw bone for the purpose of inserting a tooth implant into the jaw bone or for introducing a pilot hole for a tooth implant, into the jaw bone, comprising
a set of positioning cylinders 1, designed according to the invention, for positioning ana guiding the surgical drill, and
a set of surgical drills for drilling the hole,

wherein
the positioning cylinders 1 of a set nave an identical axial thickness L8 of the cylinder base 8 at the proximal, end 6, and
the drills of a set have defined and mutually different drilling length limiters, resulting in graduated drilling lengths of the drill,
and the depth of the hole drilled in the bone is the result of the drilling depth of the drill used from the set of drills minus the axial thickness L8 of the cylinder base 8.

If the positioning cylinder 1 in the drilling jig is screwed basally in the direction of bone contact, and if the positioning cylinders of a set have an identical axial thickness L8 of the cylinder base 8 at the proximal end 6, that is to say a specific predetermined dimension, for example of 3 mm the depth of the hole drilled in the bone is determined by the length of the drill which is used from the drill set and with which the drilling is carried out. The length of the drill is chosen accordingly. The drilling depth and the axial, thickness L8 of the base together define the drill length usable by the drilling length limiter. The depth of the hole drilled in the bone is therefore determined by the use of different drill lengths with a constant thickness of the cylinder base 8. The axial lengths L2 of the cylinder bodies 2 can be different and can be chosen according to the requirements, without this having any influence on the drilling depth. The user is in this way provided with a very clearly structured system of positioning cylinders and associated drills.

The graduation of the drilling lengths of the drills of a set is advantageously between 0.1 mm and 5.0 mm, preferably between 0.2 mm and 3 mm, particularly preferably between 0.3 mm and 1.5 mm, more preferably between 0.5 mm and 1.0 mm. The drilling lengths of the drills of a set are advantageously between 5.0 mm and 60.0 mm, preferably between 10 mm and 50 mm, particularly preferably between 15 mm and 40 mm. Preferred diameters of drills are between 0.8 mm and 5.0 mm, preferably between 1.0 mm and 2.5 mm. It is also advantageous if the drills of a set have a graduated diameter, preferably in steps of 0.1 mm.

### LIST OF REFERENCE SIGNS

1 positioning cylinder

2 cylinder body

3 inner bore

4 outer thread

5 tool socket

6 proximal end

7 distal end

6 cylinder base

9 longitudinal slot

10 flanks

11 thread tooth

12 flat

L2 length of 2

D2 external diameter of 2

D3 diameter of 3

d3 diameter of 3

L8 serial thickness of 8

D9 width of 9

19 length of 9

B12 width of 12

W flank angle

1. A positioning cylinder that can be screwed into a drilling jig in order to position and guide a surgical drill when drilling a hole in a bone, in particular in a jaw bone for the purpose of inserting a tooth implant into the jaw bone or for introducing a pilot hole for a tooth implant into the jaw bone, comprising
a cylinder body that extends in an axial direction
and that has a continuous axial inner bore and
an outer thread for screwing the positioning cylinder into a drilling jig,
characterized in that
the positioning cylinder is designed as a one-piece or integral part, and
the inner bore is used to guide the drill.

2. The positioning cylinder as claimed in claim 1, characterized in that the cylinder body does not comprise any spacer element inserted therein.

3. The positioning cylinder as claimed in claim 1, wherein the cylinder body has a constant external diameter along its entire length.

4. The positioning cylinder as claimed in claim 1, wherein the cylinder body has a constant external diameter along its entire length, except for a small conically tapering tip at its proximal end.

5. The positioning cylinder as claimed in claim 1, wherein the external diameter of the cylinder body is between 3 mm and 8 mm, preferably between 3.5 mm and 6 mm, particularly preferably between 4 mm and 5 mm.

6. The positioning cylinder as claimed in claim 1, wherein the axial length of the cylinder body is between 5 mm and 35 mm, preferably between 8 mm and 30 mm, particularly preferably between 10 mm and 25 mm.

7. The positioning cylinder as claimed in claim 1, wherein the cylinder body has, at the proximal end, a cylinder base through which the inner bore for guiding the drill is routed.

8. The positioning cylinder as claimed in claim 7, wherein the cylinder base has an axial thickness of between 0.5 mm and 10.0 mm, preferably between 1.0 mm and 8 mm, particularly preferably between 1.5 mm and 5 mm.

9. The positioning cylinder as claimed in claim 7, wherein the cylinder base has an axial thickness that is between 1% and 25%, preferably between 3% and 20%, particularly preferably between 5% and 15% of the axial length of the cylinder body.

10. The positioning cylinder as claimed in claim 7, wherein the diameter of the inner bore in the cylinder base is between 0.8 mm and 5.0 mm, preferably between 0.9 mm and 3 mm, particularly preferably between 1.0 mm and 2.0 mm.

11. The positioning cylinder as claimed in claim 7, wherein the inner bore has a greater diameter at the distal end of the cylinder body than in the cylinder base.

12. The positioning cylinder as claimed in claim 11, wherein the inner bore, with the greater diameter at the distal end of the cylinder body, extends in the axial direction as far as the distal end of the cylinder base.

13. The positioning cylinder as claimed in claim 7, wherein the inner bore at the distal end of the cylinder body has a diameter that is more than 10%, 20%, 30%, 40%, 50%, 60%, 80%, 100%, 125%, 150% or 200% greater than the diameter of the inner bore in the cylinder base.

14. The positioning cylinder as claimed in claim 1, wherein the cylinder body has, at its distal end, at least one axial longitudinal slot in the wall of the cylinder body, which slot extends in the axial proximal direction from the distal end of the cylinder body.

15. The positioning cylinder as claimed in claim 14, wherein the width of the longitudinal slot is greater than the diameter of the inner bore of the cylinder body or of the inner bore in the cylinder base.

16. The positioning cylinder as claimed in claim 15, wherein the width of the axial longitudinal slot is more than 110%, 125%, 150%, 175%, 200%, 250% or 300% of the diameter of the inner bore in the cylinder base.

17. The positioning cylinder as claimed in claim 15, wherein the width of the axial longitudinal slot is less than 500%, 450%, 300%, 250% or 200% of the diameter of the inner bore in the cylinder base.

18. The positioning cylinder as claimed in claim 14, wherein the axial longitudinal slot extends along a length that is between 10% and 75%, preferably between 20% and 60%, particularly preferably between 25% and 50% of the axial length of the cylinder body.

19. The positioning cylinder as claimed in claim 14, wherein it has a plurality of axial longitudinal slots, preferably two axial longitudinal slots.

20. The positioning cylinder as claimed in claim 19, wherein the axial longitudinal slots are arranged lying opposite each other in pairs.

21. The positioning cylinder as claimed in claim 1, wherein the outer thread extends over the entire axial outer surface of the cylinder body.

22. The positioning cylinder as claimed in claim 1, wherein the external diameter of the outer thread is between 0.01 mm and 1.5 mm, preferably between 0.02 mm and 1.0 mm, particularly preferably between 0.03 mm and 0.5 mm greater than the root diameter of the outer thread.

23. The positioning cylinder as claimed in claim 1, wherein the lead and/or the pitch of the outer thread is between 0.5 mm and 4 mm, preferably between 0.8 mm and 2 mm.

24. The positioning cylinder as claimed in claim 1, wherein the outer thread is a trapezoid thread.

25. The positioning cylinder as claimed in claim 24, wherein the thread turns of the trapezoid thread have flanks shaped as pointed cones.

26. The positioning cylinder as claimed in claim 25, wherein the flanks shaped as pointed cones have a flank angle of between 40° and 80°, preferably between 50° and 70°, with respect to each other.

27. The positioning cylinder as claimed in claim 24, wherein the thread teeth have a flat.

28. The positioning cylinder as claimed in claim 27, wherein the flat has a width of between 0.03 mm and 0.2 mm, preferably of between 0.05 mm and 0.1 mm.

29. The positioning cylinder as claimed in claim 1, wherein it is made of plastic.

30. The positioning cylinder as claimed in claim 1, wherein it is made of industry steel no. 14301, 14303 or 14305.

31. The positioning cylinder as claimed in claim 1, wherein it is made of titanium, titanium nitrite, titanium nitride, zirconium, zirconium oxide or ceramic.

32. A system for drilling a hole in a bone using a surgical drill with a predefined drilling depth of the hole in the bone, in particular in a jaw bone for the purpose of inserting a tooth implant into the jaw bone or for introducing a pilot hole for a tooth implant into the jaw bone, comprising
a set of positioning cylinders as claimed in claim 1, for positioning and guiding the surgical drill, and
a set of surgical drills for drilling the hole,
wherein
the positioning cylinders of a set have an identical axial thickness of the cylinder base at the proximal end, and
the drills of a set have defined and mutually different drilling length limiters, resulting in graduated drilling lengths of the drill,
and the depth of the hole drilled in the bone is the result of the drilling depth of the drill used from the set of drills minus the axial thickness of the cylinder base.

33. The system as claimed in claim 32, wherein the graduation of the drilling lengths of the drills of a set is between 0.1 mm and 5.0 mm, preferably between 0.2 mm and 3 mm, particularly preferably between 0.3 mm and 1.5 mm, more preferably between 0.5 mm and 1.0 mm.

34. The system as claimed in claim 32, wherein the drilling lengths of the drills of a set are between 5.0 mm and 60.0 mm, preferably between 10 mm and 50 mm, particularly preferably between 15 mm and 40 mm.

35. The system as claimed in claim 32, wherein the drills have a diameter of between 0.8 mm and 5.0 mm, preferably of between 1.0 mm and 2.5 mm.

36. The system as claimed in claim 32, wherein the drills of a set have a graduated diameter, preferably in steps of 0.1 mm.
